(19) United States
(12) Patent Application Publication
(10) Pub. No.: US 2007/0049924 A1
(43) Pub. Date: Mar. 1, 2007

(54) Ablation catheter for setting a lesion

(75) Inventor: Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/515,306

(22) Filed: Aug. 31, 2006

(30) Foreign Application Priority Data
Sep. 1, 2005 (DE) 10 2005 041 601.2

(51) Int. Cl. A61B 18/14

(52) U.S. Cl. 606/41

(57) ABSTRACT

Ablation catheter for setting a lesion, which catheter contains an ablation element that can be slid out of a catheter sleeve and has a looped section which, when said element is slid out, will self-expand into an automatically or manually imposed pre-specified shape corresponding to the actual shape of the area of tissue requiring to be ablated.

### CROSS REFERENCE TO RELATED APPLICATIONS

This application claims priority of German application No. 10 2005 041 601.2 filed Sep. 1, 2005, which is incorporated by reference herein in its entirety.

### FIELD OF THE INVENTION

The invention relates to an ablation catheter for setting a lesion.

### BACKGROUND OF THE INVENTION

During electrophysiological procedures, one or more catheters are inserted into anatomical regions of the heart for the purpose of ablating, which is to say obliterating intracardial tissue. Ablation is performed with the aid of an ablation catheter and serves to permanently treat instances of arrhythmia. Ablating in the vicinity of high-risk areas does, though, pose a risk for the patient that cannot be disregarded, namely of sustaining undesired irreparable injuries from said ablating. So when ablation is performed on atrial fibrillation in the left atrioventricle, for example, the pulmonary veins leading into the left atrium are nowadays no longer isolated by means of circular lesions in the area of the ends of said veins as that would entail a relatively high risk of producing stenoses of the pulmonary veins; ablation is instead performed so as to produce a linear lesion in the left atrium's “antrum” further away from the ends of the pulmonary veins, the aim of which lesion is likewise to electrically isolate the pulmonary veins.

Planning where to produce said lesions as well as the shape they are to have are highly dependent on individual patients' specific anatomy, because the anatomy of the left atrium in terms of its shape and the number and nature of the ends of the pulmonary veins (as a rule four or five pulmonary veins with, in part, shared ends) varies greatly from person to person.

Producing the linear lesion through ablation is also a very time-consuming process that is difficult to perform. Each linear lesion is today produced by means of a sequence of individual punctiform ablations, with each ablation site having to be traveled to separately via the ablation catheter. Thus an atrial fibrillation ablation takes about three hours to complete. What is also problematic is carrying out respective local ablating to a sufficient extent; ensuring, that is to say, that the intracardial tissue will have been obliterated sufficiently to effect the desired electrical isolation in that area. That is because owing to the patient-specific geometry of the atrioventricle or, as the case may be, irregular three-dimensional surface contours, and the fact that each point has to be traveled to separately with the ablation catheter, there is no assurance that the ablation catheter will in each instance be positioned correctly relative to the tissue or that the desired or, as the case may be, necessary degree of obliteration will be achieved during ablation. As traveling to the correct, predetermined ablation location is also difficult, there is no assurance, either, that the individual ablation points will actually be set at the correct site and be spaced apart such as actually to produce complete isolation. Albeit the ablation catheter's motion is continuously monitored during ablation, for example through x-ray monitoring, it is nevertheless extremely difficult to produce the lesion using the ablation catheter operating point-by-point.

### SUMMARY OF THE INVENTION

The problem underlying the invention is hence to disclose an ablation catheter that displays improvements on the above type and will allow a lesion to be set more easily.

Said problem is resolved by providing an ablation catheter for setting a linear lesion, which catheter includes an ablation element that can be slid out of a catheter sleeve and has a looped section which, when said element is slid out, will self-expand into an automatically or manually imposed pre-specified shape corresponding to the actual shape of the area of tissue requiring to be ablated.

The invention proposes using a looped ablation catheter whose looped section, by means of which ablation takes place, has an imposed pre-specified shape that has been imposed in advance in keeping with the actual shape of the ventricle surface in the ablation area. Said looped section is initially located inside the catheter sleeve. The ablation element with the front looped section will be pushed out of the catheter sleeve once the catheter has been pushed into the ventricle, with said looped section automatically expanding or, as the case may be, opening out and assuming the imposed pre-specified shape. The doctor can then move the looped ablation section, pre-shaped to suit the individual patient, to the correct location at which, as determined in advance while treatment was being planned, the lesion is to be produced. Owing to its three-dimensionally pre-specified shape, the looped section will be positioned against the tissue precisely in the area along which the lesion is to be produced.

Greatly simplified ablating is facilitated thereby. The doctor is required simply to correctly position the catheter once; awkward traveling to the individual ablation locations, as was hitherto necessary, is totally dispensed with. Because the shape of the looped ablation section has been matched to the ablation area's three-dimensional shape, it is furthermore assured that the positional relationship will be precise and, consequently, that ablating can take place everywhere with the required intensity. The ultimate consequence of this is that ablating, and hence setting of the linear lesion, can be carried out much faster now that the complex handling operations involved in repeated catheter positioning are no longer required. For patients this means their treatment will be much quicker and less stressful; furthermore, a successful treatment can be achieved much more reliably because the difficulties described in the introduction will no longer exist owing to shape matching.

According to a first inventive alternative the looped section can be a wire over whose entirety, which is to say along whose entire length, the high-frequency energy supplied from a coupled or couplable HF source can be conveyed into the tissue. That means the wire section will, along its defined length, obliterate the tissue immediately adjacent to it. It is alternatively conceivable for a plurality of separate ablation means, via which the high-frequency energy supplied from the coupled or couplable HF source is conveyed into the tissue, to be provided distributed on the looped wire section. In contrast to the above-described implementation variant, point-by-point ablating is carried out here and not end-to-end obliterating in a line. Since, though, the punctiform ablation means are arranged on the wire section in a stable manner they have, as a result, a defined mutual spacing so that, in conjunction with the correct fit on the tissue due to three-dimensional shaping, they will enable defined ablation points to be produced having a sufficient density to realize complete electrical isolation.

The looped wire section serving as a HF-conveying means can consist of a plurality of separate wire segments over which the HF energy can be conveyed separately. This means the HF energy can be conveyed sequentially over the individual subsegments so that the individual lesion sections are produced sequentially. Said energy can alternatively also be conveyed simultaneously. Embodying the section in the form of a plurality of subsegments of course also offers the possibility of producing the lesions in certain areas only, even with the entire looped wire section fitting along its entire length against the tissue completely and closely.

HF energy can in a corresponding manner also be applied sequentially separately to the individual, separate punctiform ablation means provided on the loop section, or simultaneously.

According to one embodiment of the invention, as an alternative to employing an ablation wire it is also conceivable to use a tube to form the looped section, through which tube a cryogen can be ducted from a coupled or couplable cryogen source. The alternative to employing the entire tube or, as the case may be, the entire length thereof to form the lesion provides here also for providing on the looped section a plurality of separate ablation means for forming ablation points, which means can be supplied with cryogen from a coupled or couplable cryogen source. The ablation catheter is in this embodiment of the invention embodied for producing a cryolesion; it is therefore a cryoablator with which the tissue is ablated by means of the cold conveyed via the cryogen. The cryogen is ducted to the tube or the separate cryoablation means by a pump via a suitable feeder line or via separate feeder lines.

Here, too, it is conceivable for the looped tube to be formed from a plurality of separate tube segments to which cryogen can be ducted separately so that individual lesions can also be set here locally. The tube segments can be supplied with cryogen sequentially or simultaneously. The same applies analogously to the individual ablation means.

An especially advantageous embodiment of the invention provides for providing on the looped section, however formed, one or more electrodes for deriving electrophysiological signals over a signal lead on the catheter side. An intracardial ECG, for instance, can be derived via said measuring electrodes. The looped section's necessary wall contact can also be checked via these immediately prior to ablation, meaning, therefore, that correct positioning can also be checked electrophysiologically. The ablation section or the segments or the individual ablation elements can thus be activated precisely when the assigned electrodes or, as the case may be, signals received indicate a good wall contact.

Alongside the ablation catheter itself the invention further relates to a method for producing an ablation catheter of such kind. This is produced by determining the three-dimensional surface contours at the ablation site using a set of 3D image data recorded pre-operatively, then distorting the ablation element's looped section accordingly for imposing the pre-specified shape. The surface contours are preferably determined automatically, for which purpose the contours of both sides of the lesion requiring to be set are defined, in particular marked, in, for example, a two- or three-dimensional representation of the ablation area on a monitor, after which the surface contours along the defined lesion are determined automatically using the set of 3D image data.

The determined data describing the surface contours can then be conveyed to a device for forming the looped section, which device will automatically impose the pre-specified shape on the section as a function of the pre-specified data.

The endocardial surface of the ventricle requiring treatment is therefore extracted, by means of, for example, segmenting, with the aid of, in particular, a three-dimensional representation on a monitor from a pre-procedurally recorded three-dimensional set of image data. The area requiring treatment, that is to say, for example, the endocardium, is displayed, together with ends of vessels or other high-risk areas, using suitable visualizing (fly-through visualizing, for example). The linear lesion requiring to be set is marked on the display by the planning electrophysiologist as a 3D line, for which purpose appropriate work tools are provided on the computing device that serves to perform planning. The 3D data of the marked line is registered automatically on the computer side and stored in world coordinates, which is to say as dimensions corresponding to the actual anatomy, and used as data for the ensuing shaping step.

The patient-specific catheter's looped section is produced in said ensuing shaping step in keeping with the planned 3D line using the three-dimensional line data. It must at this point be noted that a plurality of independent 3D lines can of course also be marked in a three-dimensional representation of the area being treated and used to produce separate catheter sections or, as the case may be, ablation sections. The shape can be imposed on the wire automatically using a suitable pressing or bending device. On completion of the three-dimensional shaping step the ablation element will be slid into the catheter sleeve along with the looped ablation section, which folds up in the process. Only when the ablation site has been reached will the section be slid out of the sleeve, then opening out automatically and assuming the pre-specified patient-specific or vessel-specific shape.

As soon as the ablation element has assumed the 3D shape the section will be ducted under realtime imaging control and so placed in position exactly as provided when treatment was planned. It is for this purpose important for both the ablation section, or parts thereof, and all major anatomical structures, or parts thereof, such as, for example, the endocardium of the ventricle being treated, the ends of the pulmonary veins, high-risk areas etc., to be visualized together with the aid of realtime imaging. Two-dimensional x-ray monitoring or intracardial 2D or 3D ultrasound, or a combination of said imaging modalities, can be employed for realtime imaging. Pulmonary vein angiograms are advantageously produced when 2D x-ray imaging is used so that the ends of the pulmonary veins can be visualized and the shaped ablation wire placed in position relative to said ends of the pulmonary veins. Just prior to actual ablation, which is to say when correct positioning has taken place, the ablation section's correct positioning can be checked and, where applicable, corrected with the aid of, for instance, a final 3D C-arc x-ray rotation angiogram, also, where applicable, in conjunction with the signals registered via the measuring electrodes. It is also conceivable for the realtime image data mentioned to be overlaid with the pre-operatively recorded three-dimensional image data (from a CT examination, for example) used for planning. Said overlaying will make it possible to verify the ablation section's actual position relative to the planned lesion (contained in the pre-operatively recorded 3D image data as a result of marking by the electrophysiologist).

### BRIEF DESCRIPTION OF THE DRAWINGS

Further advantages, features, and details of the invention will emerge from the exemplary embodiments described below and with the aid of the drawings.

FIG. 1 is a schematic of an inventive ablation catheter having an ablation element retracted into the catheter sleeve,

FIG. 2 shows an ablation catheter illustrated in FIG. 1 having an ablation element that has been slid out and has opened out,

FIG. 3 is a schematic of a three-dimensional view of the ablation area with a linear lesion marked, and shows the implementation thereof for shaping the ablation element,

FIG. 4 shows a further inventive embodiment variant of an ablation catheter having local ablation means,

FIG. 5 shows the ablation catheter illustrated in FIG. 4, augmented to include the electrodes located on the ablation element, and

FIG. 6 shows a further embodiment variant of an inventive ablation catheter having an ablation element consisting of a plurality of segments.

### DETAILED DESCRIPTION OF THE INVENTION

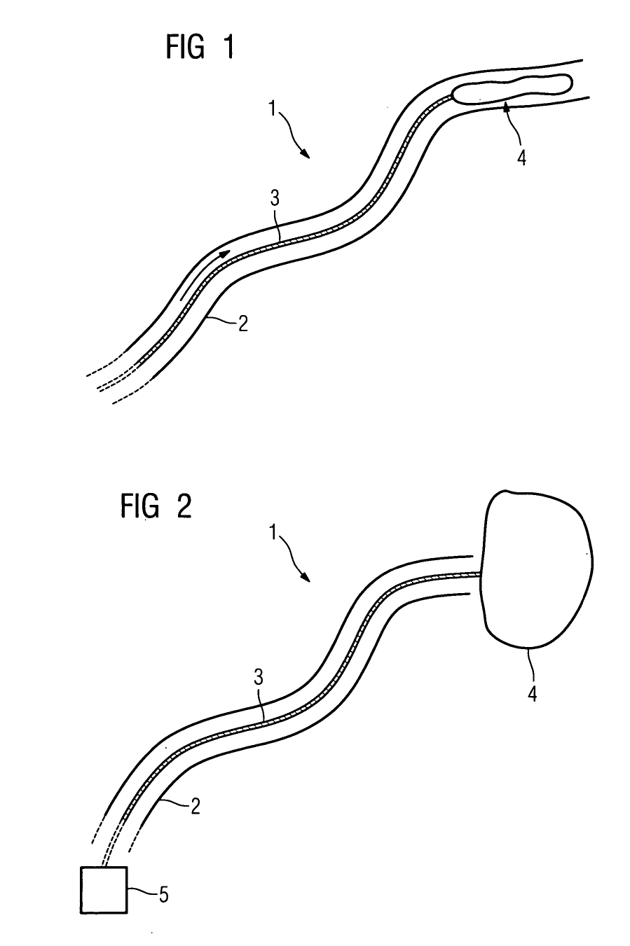

FIG. 1 shows schematically an inventive ablation catheter 1 consisting of the catheter sleeve 2 inside which is ducted a wire ablation element 3. Said ablation element 3 has on its front end a looped section 4 which, in the example shown, consists likewise of a thin wire. Said wire or, as the case may be, said section 4 can be folded so that it can be retracted inside the catheter sleeve 2.

The section 4 will be slid out of the catheter sleeve when the wire ablation element 3 is slid forward in the direction of the arrow shown in FIG. 1 after the catheter has been inserted into, for example, the ventricle. The resilient wire section 4 will unfold in the process and assume a closed shape imposed on it in advance, as shown in FIG. 2. Said shape will thus match as closely as possible that of the area of tissue on which ablating is to be performed, for instance the area around the pulmonary veins. That means the imposed shape of the wire section 4 can ultimately be of any kind, meaning it can exhibit any kind of irregular three-dimensional geometry, but is matched as closely as possible to the actual anatomical shape of the section of tissue being treated. The ablation catheter 1 will then in its opened-out condition continue being moved under x-ray realtime control, or suchlike, until the wire section 4 in positioned precisely where the lesion is to be produced, which is to say is positioned, owing to its three-dimensional anatomically matched shape, precisely against the section of tissue whose shape it maps. Through having been shaped, it will consequently fit closely and precisely against the tissue requiring to be ablated. The high-frequency energy (HF energy) required for ablating is then coupled into the ablation wire via, for instance, a control device 5, which is coupled to the ablation element 3 and constitutes a high-frequency source (HF source), and conveyed over the wire ablation section 4 into the adjacent tissue, which will be obliterated via said section. A linear lesion can thus be produced along the section 4 by supplying HF energy once, which means the wire section 4 serves here in its entirety to form the lesion.

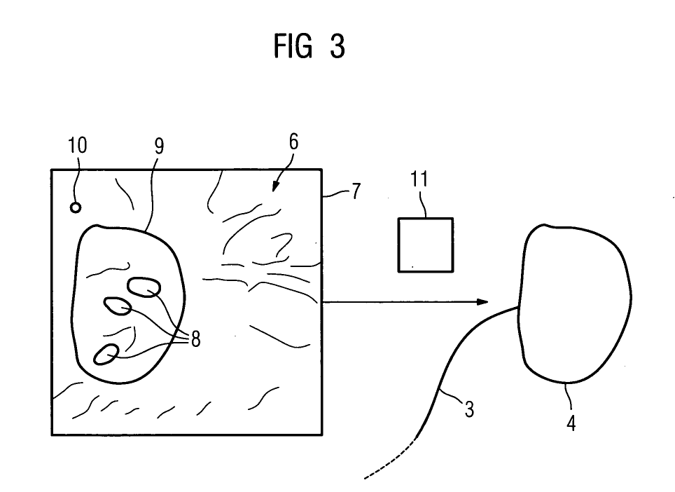

FIG. 3 shows schematically the procedure for imposing the pre-specified shape on the wire section 4.

A three-dimensional, where applicable previously segmented image 6 is initially fed out on a monitor 7 based on a set of 3D image data pre-procedurally recorded via, for example, a computer tomography scanner. Said image 6 shows, in the example illustrated, the antrum of the left atrium, with, in the example shown, a view of three pulmonary veins 8 ending there. Using a suitable software processing module, the doctor or electrophysiologist can then enter a marking 9 in said three-dimensional representation marking the contours of the ablation requiring to be performed or, as the case may be, the location of the linear lesion that is to be produced for electrically isolating the three pulmonary veins 8. The assigned computer device then determines the corresponding spatial or world coordinates or, as the case may be, corresponding positional data representing the marking's three-dimensional location on the surface 10 of the atrium. With the aid of said data, which is conveyed to a suitable shaping device 11 automatically, the looped section 4 of the ablation element 3, which has been moved into said device, is then shaped, which is to say bent, accordingly. The finished ablation element consequently has a patient-specifically or, as the case may be, anatomically precisely shaped ablation section 4 corresponding exactly to the actual anatomy of the previously defined ablation area on the section of tissue. It must at this point be noted that instead of being shaped mechanically the ablation section 4 can, of course, also be shaped manually if the three-dimensional section shape requiring to be formed is visualized to, for example, the doctor or electrophysiologist on the monitor.

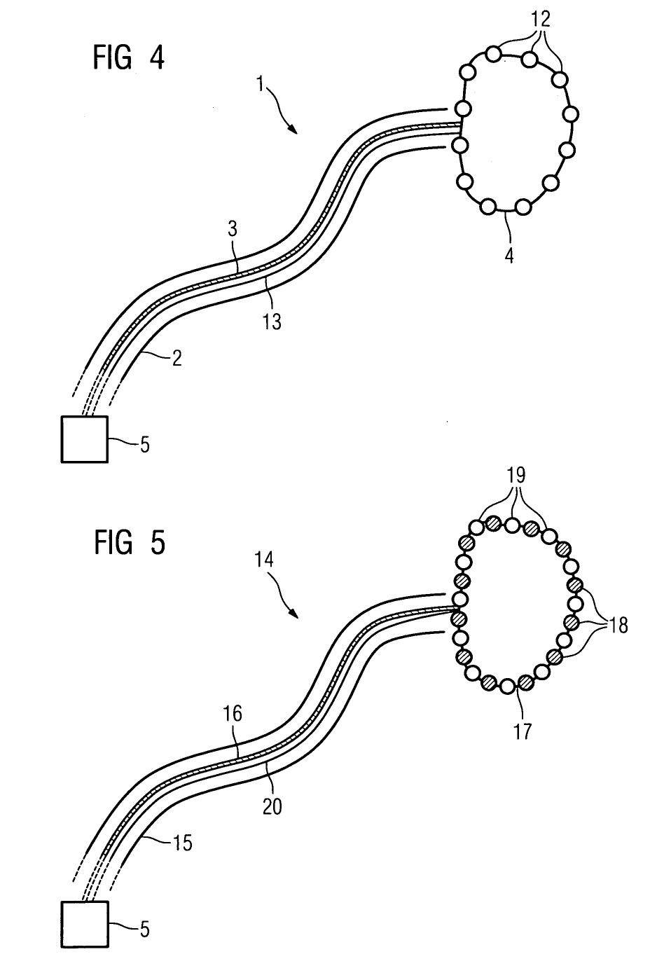

FIG. 4 shows an ablation catheter 1, already known from FIG. 1, comprising the catheter sleeve 2 and the integrated ablation element 3 having the looped ablation section 4. In addition to the embodiment according to FIG. 1, arranged on the looped section 4 are a series of individual electrodes 12 for deriving electrophysiological signals via a signal lead 13 that is additionally ducted on the catheter side and via which the electrode signals, individually resolved, are conveyed externally to the control device 5, that also serves to perform signal processing. The looped section's wall contact can be checked via said electrodes 12 so that ablating will not take place, which is to say the HF energy will not be applied, until adequate wall contact has been assured. Being located on the three-dimensional, surface-specifically shaped section 4, the electrodes 12 will consequently, if the section 4 is correctly positioned, likewise be positioned optimally against the tissue wall so that the correct wall contact and hence also the correct position can clearly be registered via their signal.

FIG. 5 shows a further embodiment variant of a catheter 14 having an ablation element 16, arranged slidably therein, having a looped section 17 that has a pre-specified, imposed shape and can be retracted in a collapsible manner inside the catheter sleeve 15. Departing from the embodiment according to FIGS. 1 to 4, the section 17 does not here itself serve to convey the supplied HF energy; a series of individual ablation means 18 distributed along the length of the section 17 are instead fixed in position, preferably mutually equidistantly, in a stable manner, and between them, in the example shown, are arranged corresponding electrodes 19 for registering electrophysiological signals. In this embodiment the HF energy is conveyed over the individual ablation means (of which in actuality substantially more are arranged in position than are shown in FIG. 5). The individual ablation elements are powered via the wire feeder, with is to say via the ablation element 16 itself.

The respective connection between the individual ablation means 18 and the energy feed can be such that all ablation means 18 can be supplied with HF energy simultaneously, meaning that ablating can take place via all ablation means 18 at the same time. It is alternatively also conceivable for the line connection to the individual ablation means 18 to be embodied such that the ablation means 18 can be supplied with HF energy separately, where applicable also in groups, so that ablating can take place, as it were, sequentially from ablation means 18 to ablation means 18.

Here, too, it is possible via the intermediately arranged electrodes 19 to establish optimal positioning of the section 17 and hence of the ablation means 18 with reference to the tissue wall, with signals being conveyed over a corresponding signal lead 20 inside the catheter sleeve to the external control device 5 in this case, also.

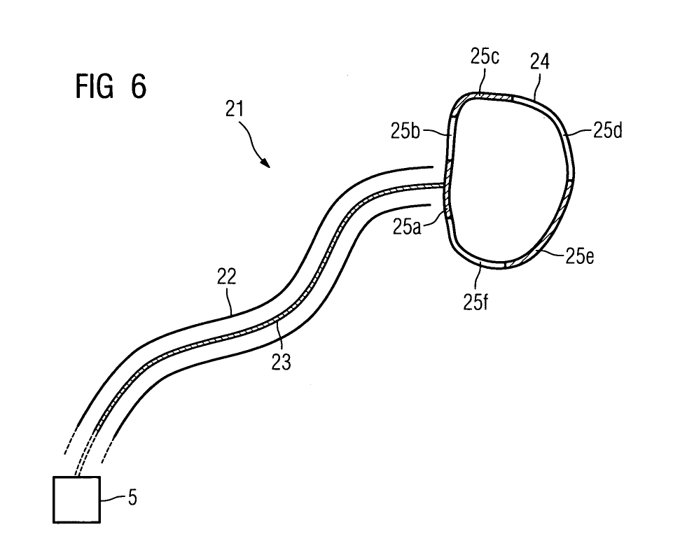

Finally, FIG. 6 shows a further inventive embodiment variant of an ablation catheter 21 comprising a catheter sleeve 22 having, arranged slidably therein, a wire ablation element 23 which likewise has a looped section 24 that can be folded and retracted into the catheter sleeve 22 and slide out of it assuming a pre-specified, three-dimensional, shape-matched tissue surface shape. Here, too, the resilient section 24 itself serves to convey the coupled HF energy, which is to say to set the linear lesion. Departing from the embodiment variant according to FIG. 1, the looped section 24 is here assembled from a multiplicity of individual wire segments 25a to 25f. These are mutually isolated and can be supplied separately with HF energy over the wire feeder, formed via the wire part of the ablation element 23 ducted in the catheter sleeve 22, for which purpose corresponding line connections are provided. This means that the wire section inside the catheter sleeve can consist of a plurality of individual strands each leading to in each case one wire segment 25a to 25f. Said multi-stranding is of course also possible in the case of the previously described embodiment shown in FIG. 5 having the individual ablation means 18.

The section 24 is in any event shaped here, too, in keeping with the three-dimensional surface shape of the tissue section being treated. Departing from what is the case with the single-piece section, the lesion can here be produced by sequentially producing individual partial lesions which in their totality will then form the linear lesion.

Regardless of how the ablation catheter is specifically embodied, the ablation treatment requiring to be carried out therewith will proceed essentially in six steps.

In the first step a three-dimensional representation of the segmented surface requiring treatment, for example the ventricle being treated, is displayed on a monitor, which representation is obtained using a pre-procedural set of 3D image data recorded in advance by means of, for instance, a CT scanner. The linear lesion requiring to be planned is then marked as a 3D line in said representation by the doctor providing the treatment and the shape of said 3D line determined on the computer side as corresponding positional data and stored. A plurality of separate lesions can, of course, also be marked as part of this process and their positional data determined.

In the second step the ablation element is produced, or, as the case may be, its front section is shaped patient-specifically. The ascertained 3D positional data from the first step provides the basis for this. Said data can be conveyed electronically directly from the computer device ascertaining it to a device that will shape the section, which device will then automatically re-shape the section or, as the case may be, impose the shape. The catheter element is then inserted into the catheter sleeve, with the pre-formed section folding up and disappearing, likewise, inside the catheter sleeve.

In the third step the catheter is ducted into the ventricle undergoing treatment, after which, in the fourth step, once the catheter has been placed in position in said ventricle, the ablation element is slid forward out of the catheter sleeve, specifically to an extent that the section protrudes from the sleeve completely and automatically opens out into the predetermined three-dimensional shape.

In the fifth step the section is positioned under the control of realtime imaging (2D x-ray or intracardial 2D or 3D ultrasound) in such a way that it will fit along its entire length against the tissue wall, meaning it will be positioned so as to be precisely integrated into the area of tissue. All necessary anatomical structures in the area undergoing treatment are for this purpose visualized to the doctor by means of the realtime imaging. Prior to final ablating, the correct positioning can be re-checked via a 3D C-arc rotation angiogram, where applicable in conjunction with the registering of electrophysiological signals via electrodes on the section side.

Actual ablation then takes place in the sixth and final step.

It must in conclusion be noted that, instead of the possibility shown in FIGS. 1 to 6 of ablating through conveying HF energy, cryoablation is also possible. In that case the respective resilient section 4, 17, or 24 in the catheter embodiments described would consist of a single-piece resilient tube or (in the case of section 24) of tube segments through which it is possible to convey a cryogen that can be fed via the respective ablation element's likewise tubular feeder section ducted in the catheter sleeve. In the case of a segment-type structure a plurality of such tubular feeder lines, one of which leads in each case to a tube segment, can also be provided in the sleeve for supplying the individual segments separately. Here, too, cryogens are supplied via, for example, the central control device 5. Cryoablation means to which cryogen can be supplied would in this case be used instead of the HF ablation means 18.

1-15. (canceled)

16. An ablation catheter for setting a lesion of a tissue to be ablated of a patient in a medical procedure, comprising:
a catheter sleeve;
an ablation element that is enclosed in the catheter sleeve and has a front end; and
a looped section that is arranged on the frond end of the ablation element and self-expanded into an imposed pre-specified shape corresponding to an actual shape of an area of the tissue when the ablation catheter is inserted into the area and the ablation element is slid out of the catheter sleeve.

17. The ablation catheter as claimed in claim 16, wherein the looped section is a wire.

18. The ablation catheter as claimed in claim 17, wherein the entire looped wire section conveys high frequency energy supplied from a coupled high frequency source and obliterates the tissue in a line.

19. The ablation catheter as claimed in claim 17, wherein a plurality of separate ablation configurations is distributed along the looped wire section conveying high frequency energy supplied from a coupled high frequency source and obliterates the tissue point-by-point.

20. The ablation catheter as claimed in claim 19, wherein the separate ablation configurations are supplied with the high frequency energy sequentially or simultaneously.

21. The ablation catheter as claimed in claim 17, wherein the looped wire section comprises a plurality of separate wire segments over which high frequency energy supplied from a coupled high frequency source is conveyed separately.

22. The ablation catheter as claimed in claim 21, wherein the separate wire segments are supplied with the high frequency energy sequentially or simultaneously.

23. The ablation catheter as claimed in claim 16, wherein the looped section is a tube.

24. The ablation catheter as claimed in claim 23, wherein the entire looped tube section is ducted with a cryogen from a coupled cryogen source and obliterates the tissue in a line.

25. The ablation catheter as claimed in claim 23, wherein a plurality of separate ablation configurations is distributed along the looped tube section supplied with a cryogen from a coupled cryogen source and obliterates the tissue point-by-point.

26. The ablation catheter as claimed in claim 25, wherein the separate ablation configurations are supplied with the cryogen sequentially or simultaneously.

27. The ablation catheter as claimed in claim 23, wherein the looped tube section comprises a plurality of separate tube segments through which a cryogen is fed separately.

28. The ablation catheter as claimed in claim 27, wherein the separate tube segments are supplied with the cryogen sequentially or simultaneously.

29. The ablation catheter as claimed in claim 16, wherein an electrode is provided on the looped section which derives an electrophysiological signal over a signal lead to a signal processing unit of the catheter for checking a wall contact between the looped section and a wall of the tissue.

30. The ablation catheter as claimed in claim 16, wherein the looped section is folded and retracted inside the catheter sleeve.

31. The ablation catheter as claimed in claim 16, wherein the pre-specified shape is imposed automatically or manually.

32. A method for setting a lesion of a tissue to be ablated of a patient in a medical procedure using an ablation catheter, comprising:
providing a catheter sleeve for the ablation catheter;
enclosing an ablation element in the catheter sleeve, the ablation element having a front end connected to a looped section;
sliding the ablation element out of the catheter sleeve after inserting the ablation catheter into an area of the tissue; and
self-expanding the lopped section into an imposed pre-specified shape corresponding to an actual shape of the area of the tissue.

33. A method for making an ablation catheter having an ablation element for setting a lesion of a tissue to be ablated of a patient in a medical procedure, comprising:
recording a set of three-dimensional image of the tissue prior to the medical procedure;
determining a three-dimensional surface contour of the tissue based on the set of three-dimensional image; and
distorting a looped section connected at a front end of the ablation element according to the surface contour for imposing a pre-specified shape of the tissue.

34. The method as claimed in claim 33,
wherein a representation of an area of the tissue is displayed on a monitor based on the set of three-dimensional image,
wherein the lesion is defined according to the representation,
wherein the surface contour along the defined lesion is automatically determined with data,
wherein the data is conveyed to a shaping device for forming the looped section, and
wherein the shaping device automatically imposes the pre-specified shape on the looped section as a function of the data.
